(19) United States
(12) Patent Application Publication
(10) Pub. No.: US 2022/0395174 A1
(43) Pub. Date: Dec. 15, 2022

(54) APPARATUS AND METHOD FOR MEASURING AT LEAST ONE VISUAL REFRACTION FEATURE OF A SUBJECT

(71) Applicant: Essilor International, Charenton-le-Pont (FR)

(72) Inventors: Gildas MARIN, Charenton-le-Pont (FR); Guillaume GIRAUDET, Charenton-le-Pont (FR)

(73) Assignee: Essilor International, Charenton-le-Pont (FR)

(21) Appl. No.: 17/763,060

(22) PCT Filed: Sep. 24, 2020
(86) PCT No.: PCT/EP2020/076802

§ 371 Date: Mar. 23, 2022

(30) Foreign Application Priority Data
Sep. 25, 2019 (EP) 19306196.7

(51) Int. Cl. A61B 3/032; A61B 3/00; A61B 3/11; A61B 3/028

(52) U.S. Cl. CPC A61B 3/032; A61B 3/0033; A61B 3/112; A61B 3/0285

(57) ABSTRACT

An apparatus and method for determining at least one visual refraction feature of a subject by showing a visual stimulus to the subject. The apparatus includes an optical system arranged on an optical path between an eye of the subject and the visual stimulus, the optical system being adapted to provide an optical power that is continuously variable as a function of time (t), a control unit for driving the optical power of the optical system and an input device adapted for recording a response of the subject relative to a sharpness of the visual stimulus seen through the optical system, the control unit being adapted to adjust a speed of variation of the optical power (S) as a function of the response recorded.

### TECHNICAL FIELD OF THE INVENTION

The invention relates to a method and apparatus for measuring refraction of the eye(s) of a subject.

More precisely the invention relates to an apparatus for measuring at least one visual refraction property of a subject, the apparatus having an optical system with an optical power that may be varied continuously. The invention also relates to a method for determining refraction properties of the eye(s) of a subject using this apparatus.

BACKGROUND INFORMATION AND PRIOR ART Numerous documents describe devices and methods for determining objectively or subjectively visual refraction features of the eyes of a subject in monocular or binocular vision conditions.

Objective refraction is usually obtained using a skiascope or an auto-refractometer. Objective refraction enables to determine quickly the approximate correction for a subject.

However, subjective refraction is the method of choice to determine the best visual refraction correction needed by a subject.

A well-known subjective-type apparatus is the Badal optometer, which uses a movable optical target, than can be moved by the user to determine his/her refraction subjectively. A lens is placed between the visual target and the eye, with the focal point in the eye pupil, so that image size does not change when the target moves.

A phoropter is another well-known instrument for subjective determination of the refraction correction needed for a subject. A phoropter generally comprises a set of optical lenses of variable optical power and a lens changer to enable testing vision of the subject. An optometrist drives the changes in lens power step by step and the subject determines subjectively the best vision correction for his/her eyes. When varying the optical power of the lens during a refraction process, it is always going from one corrective dioptric power to another such that the subject can compare both states and choose which one is the most appropriate. These devices generally use lenses of varying optical power by steps of 0.25 diopter (D). However, the subject does not perceive the visual stimulus during lens changes.

Document WO 2017/013343 A1 (Essilor International) describes a vision-compensating device comprising a lens having a variable spherical power and an optical assembly generating a variable cylindrical correction. Document US 2004/100617 A1 discloses an apparatus for interactive optometry. Document US 2015/216411 A1 discloses methods and devices for interactive adjustment of a continuously variable optical lens.

Such devices allow ensuring a smooth and continuous perception of the visual stimulus during refraction changes.

However, there is a need to keep control of accommodation during the refraction measurement process especially during a self-refraction process.

Also, there is a need of a new refraction process and apparatus taking advantages of continuous perception that help to simplify the process for self-refraction and/or speed-up the process.

### SUMMARY OF THE INVENTION

Therefore one object of the invention is to provide an apparatus and method for determining at least one visual refraction feature of a subject by showing a visual stimulus to the subject.

According to the invention, the apparatus comprises an optical system arranged on an optical path between an eye of the subject and the visual stimulus, the optical system being adapted to provide an optical power that is continuously variable as a function of time (t), a control unit for driving the optical power of the optical system and an input device adapted for recording a response of the subject relative to a sharpness of the visual stimulus seen through the optical system, the control unit being adapted to adjust a speed of variation of the optical power (S) as a function of the response recorded.

According to the present disclosure, the speed of variation is lower than a limit in perception of optical power variation of the eye of the subject and the amplitude of variations in optical power feature(s) are above a limit perception of optical power variation of the eye of the subject.

The proposed solution adjusts at least one optical power feature continuously as a function of time while adjusting the speed of the optical power variations as a function of the subject's response(s).

This technical solution enables to choose the best variation parameters to optimize perception and minimize accommodation impact. Variations in optical power feature(s) may be adapted following a periodic (or pseudo-periodic or non periodic) time function that may be adapted to the subject or some control condition of the measurement while subject determines which point is the correct one. Alternatively or complementarily, variations in optical power feature(s) may be adapted in amplitude to optimize the perception of the variations in optical power feature(s).

Rather than targeting a new power from an initial one, the power is continuously varied and the control is set on the speed of variation and observer's response. The subject may have self-control of the speed of variation, going faster when desired or slower if needed, reversing the variation or stopping by controlling the speed from negative to positive values, including null value. This self-controlled continuous determination of eye refractive error can also be done with minimum involvement of the subject reacting to a simple question such as: “press the button when the letters are the sharpest”. The time function of variation of the lens power is then adapted to the subject's answers.

To be able to control accommodation and blur adaptation, the power is continuously varied during the whole refraction exam. This power variation is rather not periodic but pseudo-periodic and/or adapted to the subject's answer.

Preferably, the control unit is adapted to adjust the speed of variation by going through predetermined stages as a function of the response recorded.

Advantageously, the visual stimulus comprises an optotype, a Gabor patch, a sinusoidal grating, a lifestyle scene, a red/green test and/or a hybrid image.

According to a particular aspect, the speed of variation decreases continuously over time or decreases over time by discrete predetermined values of speed.

According to a particular embodiment, the control unit is adapted to drive the optical power of the optical system at an initial maximum positive value, to decrease the optical power from the initial maximum positive value to a first optical power value relative to a first sharpness of the visual stimulus, with a first speed of variation, the input device is adapted for recording a first response of the subject relative to the first sharpness and the control unit is adapted to increase the optical power to a second maximum value below the initial maximum positive value.

According to a variant of this embodiment, the control unit is adapted to decrease the optical power from the first optical power value relative to the first sharpness of the visual stimulus until a first minimum value depending on the first optical power value relative to the first sharpness of the visual stimulus, before increasing the optical power to the second maximum value.

According to another aspect of this embodiment, the second maximum value depends on the first optical power value relative to the first sharpness of the visual stimulus.

According to another aspect, the control unit is adapted to implement a continuous variation of the optical power between N successively decreasing maximum values and N successively increasing minimum values, where N is an integer comprised between 2 and 5, the speed of variation between one of said maximum values and a consecutive minimum value depending on the in-between optical power value relative to a sharpness of the visual stimulus, and the following maximum value also depending on the said in-between optical power value relative to a sharpness of the visual stimulus.

According to any of the embodiments disclosed, the optical power includes a spherical power, a cylindrical power and cylinder axis and/or an addition power and/or a binocular balance between both eyes of the subject.

According to another aspect, the calculator is adapted to determine said at least one visual refraction feature of the subject as a function of one or a plurality of responses of the subject.

According to an embodiment, the control unit is adapted to select the visual stimulus. The selected stimulus depends on the current predetermined stage of variation of the speed and/or depends on the response recorded. For instance, the optotypes, such as letters, are selected with smaller and smaller size as a function of the oscillation cycle.

According to another aspect, the input device comprises a user interface adapted to record an input parameter and the control unit is adapted to drive the speed of variation as a function of the input parameter.

Preferably, the user interface comprises a button, a dimmer, a joystick, a device adapted to record a physiological signal of the subject, a voice recognition system and/or a computer interface and/or a brain-computer interface with electrodes recording brain activity in real-time and/or an interface with a pupil measurement system or with a reaction time measurement system and/or a tracking movement or eyetracking system and/or a face or hand or body expression analyzing system.

According to an embodiment, the apparatus is adapted to record a reaction time of the subject.

According to another aspect, a range of the optical power and/or a range of the speed of variation are preselected as a function of data relative to the subject and/or as a function of distance to the visual stimulus and/or the optical power variation is periodic, pseudo periodic or non periodic.

A further object of the invention is to provide a system for measuring at least one visual refraction feature of a subject, the system comprising an apparatus according to any one of the embodiments disclosed herein and further comprising an objective refraction measurement device and/or a device for measuring micro-fluctuations of refraction of the eye, adapted for providing preliminary measurements, the control unit being adapted to define an initial profile for the speed of variation of the optical power according to said preliminary measurements.

A further object of the invention is to provide a method for determining at least one visual refraction feature of a subject, the method comprising the steps of:

a) varying continuously an optical power of an optical system in a phoropter, the optical system being arranged on an optical path between an eye of the subject and a visual stimulus,

b) recording a response of the subject to the continuous variation in optical power of the optical system, the response being relative to a sharpness of the visual stimulus seen through the optical system with continuously variable optical power,

c) adjusting a speed of variation of the optical power as a function of the response recorded, and

d) repeating the steps a) to c) until a best focus is determined.

### DESCRIPTION OF THE DRAWINGS

The following description with reference to the accompanying drawings will make it clear what the invention consists of and how it can be achieved. The invention is not limited to the embodiments illustrated in the drawings. Accordingly, it should be understood that where features mentioned in the claims are followed by reference signs, such signs are included solely for the purpose of enhancing the intelligibility of the claims and are in no way limiting on the scope of the claims.

Reference is now made to the brief description below, taken in connection with the accompanying drawings and detailed description, wherein like reference numerals represent like parts.

FIG. 1 represents schematically a top view of an apparatus for measuring at least one visual refraction feature of a subject according to the present disclosure;

FIG. 2 illustrates an exemplary method of variation of an optical parameter of a continuously variable optical system according to a first embodiment;

FIG. 3 represents an example of hybrid image formed by superimposing the low spatial frequencies of a first image and the high spatial frequencies of a second image;

FIG. 4 the low spatial frequencies of the first image used in the hybrid image of FIG. 3;

FIG. 5 the high spatial frequencies of the second image used in the hybrid image of FIG. 3;

FIG. 6 illustrates an exemplary method of variation of an optical parameter of a continuously variable optical system according to a second embodiment;

FIG. 7 illustrates an exemplary method of variation of an optical parameter of a continuously variable optical element according to a variation of the second embodiment;

FIG. 8 illustrates an exemplary method of variation of an optical parameter of a continuously variable optical element according to another variation of the second embodiment;

FIG. 9 represents schematically a block diagram of a method for measuring refraction of a subject according to the present disclosure.

### DETAILED DESCRIPTION OF THE INVENTION

In the description which follows the drawings are not necessary to scale and certain features may be shown in generalized or schematic form in the interest of clarity and conciseness or for informational purposes. In addition, although making and using various embodiments are discussed in detail below, it should be appreciated that as described herein are provided many inventive concepts that may be embodied in a wide variety of contexts. Embodiments discussed herein are merely representative and do not limit the scope of the invention. It will also be obvious to one skilled in the art that all the technical features that are defined relative to a process can be transposed, individually or in combination, to a device and conversely, all the technical features that are defined relative to a device can be transposed, individually or in combination, to a process.

### Definitions

In the present document an optical power feature of an optical system includes a spherical power, a cylindrical refraction power and a cylinder axis and/or an addition refraction power and/or a binocular balance between eyes. Binocular balance includes the adjustment of the difference (balance) between the spherical power of the two eyes, wherein both lens may be adjusted.

A continuously variable optical power feature includes an optical power feature, as defined above, that may be varied continuously as a function of time.

Device

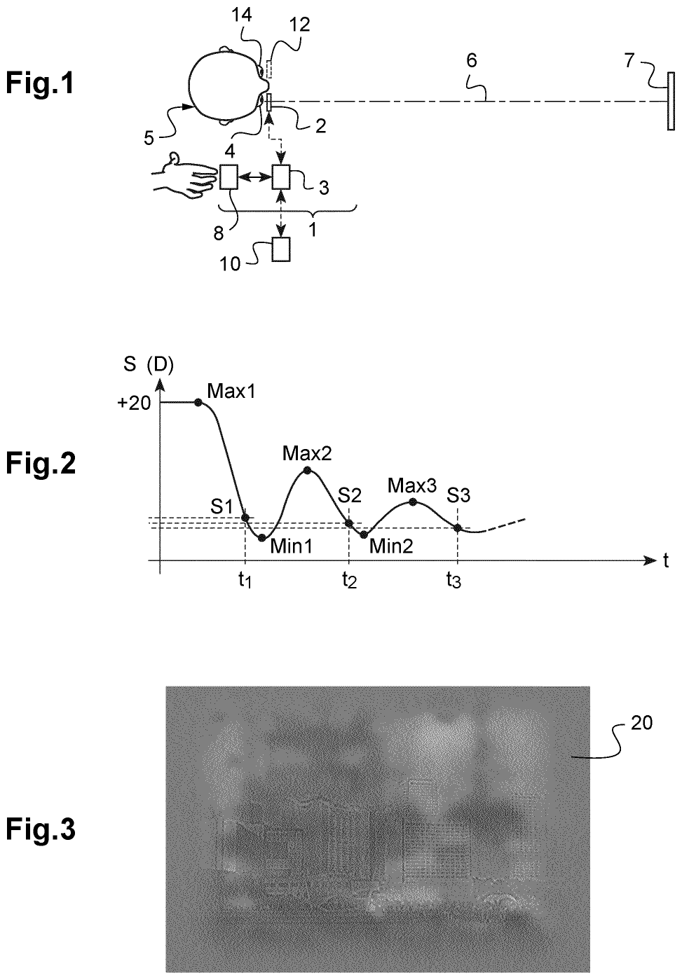

FIG. 1 represents schematically, from above, the main elements of an apparatus 1 for determining at least one refraction feature of an eye 4 of a subject 5. The eye 4 may indifferently be the right eye or the left eye of the subject 5.

The apparatus 1 comprises an optical system 2, for providing the eye 4 of the subject 5 with at least one refraction power feature. More precisely, the optical system 2 is adapted to provide a continuously variable optical power feature.

The subject 5 looks at a visual stimulus 7 through the optical system 2 that provides his/her eye 4 with an adjustable refraction correction, in order to test the subject's vision and to determine at least one refraction feature of his/her eye 4.

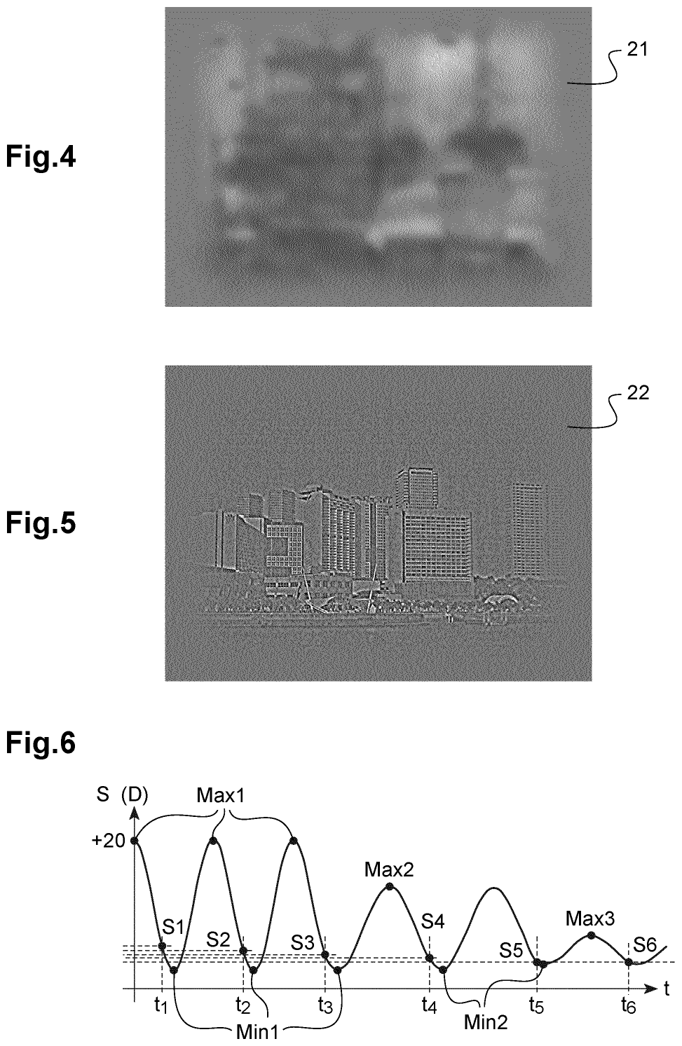

The visual stimulus 7 may be a target object, such as a screen or a panel, displaying one or several optotypes, or any image appropriate to test the vision of the subject. Alternatively, the visual stimulus 7 may be a Gabor patch, or a sinusoidal grating (with variable angle or not), or a lifestyle scene, preferably including sufficiently high spatial frequencies to help detecting blur. Alternatively, the visual stimulus 7 may be a hybrid image 20 (see FIG. 3) which superimposes a first image 21 consisting of low spatial frequencies of a scene (see FIG. 4) and a second image 22 comprising the high spatial frequencies of another scene (see FIG. 5). The visual stimulus 7 may be displayed on an image display device. The instrument 1 is configured to enable refraction measurements at various distances (near vision, far vision and/or intermediate vision) and/or for various eye gaze directions (for example natural eye gaze direction lowered for reading, horizontal eye gaze direction for far vision). The visual stimulus 7 is located at a real or virtual distance from the optical system 2 comprised between 25 cm (for near vision) and infinity (for far vision) when using a specific imaging system (not represented), such as a Badal system, or, if no imaging system is used (or using a plane mirror), up to about 8 meters in practice.

The optical system 2 is configured to provide the eye 4 of the subject with at least one continuously variable optical power feature. In the present document, a continuously variable optical power feature includes a spherical power, cylindrical power features, such as a cylindrical power and axis, an addition power and/or a binocular balance between both eyes 4, 14 of the subject 5. The continuously variable optical power feature may be varied continuously as a function of time.

To that end, a control unit 3 is connected to the optical system 2. The control unit 3 drives continuously the variable optical power, as detailed below, in order to modify the refraction correction provided to the subject in a continuous manner as a function of time, during an eye exam.

For example, the optical system 2 comprises a deformable lens, a deformable liquid lens, a multi-electrodes liquid lens based on electro-wetting, a lens based on deformable membrane(s), a lens deformable by application of hydraulic or pneumatic internal pressure, an electronically adaptive optical element, the adaptive optical element (AOE) being a transmissive AOE or a reflective AOE, a deformable mirror, a pixelated digital mirror device, a light field display device, a spatial light modulator, a liquid crystal modulator, motorized cross-cylinder lenses, a pair of Alvarez-Humphrey plates or a piezo-electric optical system.

For instance, the spherical power, noted S, of the optical system 2 is continuously adjustable as a function of time during an eye exam process. In other words, the optical system 2 is configured to provide the eye 4 with an adjustable spherical power, S, in order to determine spherical refraction correction needed for this eye 4.

A similar process applies for determining an addition power needed for correcting presbyopia in near-vision.

Alternatively or complementarily, the cylindrical power feature(s) of the optical system 2 is(are) continuously adjustable as a function of time during an eye exam process. The cylindrical power features may be decomposed in a cylinder value or cylindrical power, noted C, and in an axis, noted A, of this cylinder. Other decompositions of the cylindrical power features exist such as the couple (J0, J45). In other words, the optical system 2 is configured to provide the eye 4 with adjustable cylindrical power feature(s), in order to determine astigmatism refraction correction of this eye.

Preferably, the optical system 2 is also configured so that its optical power features can be modified without interrupting the light beam 6 that comes from the visual stimulus 7 and that is refracted or reflected by the optical system 2 to reach the eye 4 of the subject. This light beam 6 is constituted by the part of the light that comes from the visual stimulus 7 and that is collected by the optical system 2 and then transmitted to the eye 4 of the subject 5 (this light being initially emitted, diffused or reflected by the target object). The optical system 2 is thus configured so that its optical power features can be modified with no cut-off of the field of view of the eye 4 of the subject. In other words, the visual stimulus 7 remains unmasked, that is to say unclogged, to the eye 4 of the subject in the course of such an optical power feature modification.

In particular, the optical system 2 is configured so that such an optical power feature adjustment can be achieved without substituting a given lens by another (which would temporarily mask the target object to the eye of the subject).

The control unit 3 is configured to adjust the optical power feature(s) of the optical system 2 during an eye exam process using the input device 8.

The input device 8 comprises for example a commonly used computer control input device, such as a keyboard, a mouse, a button, a dimmer, a voice recognition system or a device adapted to record a physiological signal of the subject under examination, such as an electro-encephalograph delivering an electro-encephalogram (or EEG) signal, a pupil measurement system, a brain-computer interface with electrodes recording brain activity in real-time, a tracking movement or eyetracking system and/or a face or hand or body expression analyzing system or another reaction time measurement system. The subject 5 himself may manipulate the input device 8 for self-refraction. Alternatively, the input device 8 may be used by an optometrist or another person assisting the subject 5 during the eye refraction exam. Complementarily or alternatively, the input device 8 may comprise an input port of the control unit, directly connected to an output of another system for example of a computer or an electro-encephalograph system, a pupil measurement system, a brain-computer interface with electrodes recording brain activity in real-time, a tracking movement or eyetracking system and/or a face or hand or body expression analyzing system or another reaction time measurement system.

Advantageously, the apparatus 1 comprises a second optical system 12 for providing the second eye 14 of the subject 5 with a second refraction power. The second optical system 12 may be similar to the optical system 2 described above. The (first) optical system 2 and the second optical system 12 may be used alternatively while blocking or blurring the other eye path for refraction measurements in monocular vision conditions. Alternatively or complementarily, the (first) optical system 2 and the second optical system 12 may be used simultaneously for refraction measurements in binocular vision conditions, that is while the subject 5 has both eyes opened and un-obstructed, the first eye 4 of the subject 5 looking at a first visual stimulus 7 and the second eye 14 looking at a second visual stimulus. Finally, the best correction for both eyes 4, 14 may be tested in binocular vision conditions, that is while the subject 5 has both eyes opened and un-obstructed, both eyes 4, 14 of the subject 5 looking at the same or similar visual stimulus 7.

Process

FIG. 2 shows a first method of variation of an optical power feature of a continuously variable optical system. For explanation purposes, the optical system 2 has a variable spherical power S. However, a similar process may be applied to another power feature of the optical system 2 as mentioned above.

The shape of the temporal power variation shown on FIG. 2 is only for illustrative purpose. Other shapes of temporal variation may be contemplated without departing from the scope of the present disclosure.

In this example, the variable spherical power S of the optical system 2 is initially set at a first maximum value, noted Max1, that is positive, here of +20 diopters (D). Whatever the refractive state of the subject 5, the high positive value of the first maximum value, Max1, enables to initiate the eye exam with a perceived defocused stimulus and avoids any accommodation response of the subject 5.

In a first stage, the spherical power S continuously decreases from the first maximum value, Max1, at a first speed value, Speed1, of −10 D/s. In the present document, the speed value is the maximum speed value of the variation in optical power feature, here for example spherical power, between two consecutive extremum values.

The subject 5 is instructed to press a button as soon as he/she sees the visual stimulus 7 sharply through the optical system 2. Alternatively, the subject's response is formulated as an oral response that is entered by an optometrist or using a voice recognition system.

In the example of FIG. 2, the first response of the subject 5 is entered at first time instant t₁ corresponding to a first spherical power value, noted S1. The value of the first spherical power value, S1, or equivalently of the first time instant t₁, is recorded by the control unit 3.

When the first response, corresponding to the first spherical power value S1, is entered, the variable spherical power S keeps on decreasing until reaching a first minimum value, noted Min1. The first minimum value, Min1, being lower than the first spherical power value, S1, enables the subject 5 to see whether the image of the visual stimulus 7 might be even sharper below the first spherical power value, S1, or not. In the example illustrated on FIG. 2, the value of Min1 is 1 diopter (D) below the value of the first spherical power value S1.

After reaching the first minimum value, Mint, the variable spherical power S increases again up to a second maximum value, noted Max2.

In an embodiment, the second maximum value, Max2, is adjusted according to the subject's first response. For example, the second maximum value, Max2, is adjusted as a function of the first maximum value, Max1, the first spherical power value S1 and/or first minimum value Min1 according to one of the following equations:

Max2=Min1+(Max1−S1)/2

Or

Max2=(Max1+S1)/2

Thus, the second maximum value, Max2, is below the initial first maximum value Max1. The variable spherical power S increases from the first minimum value Min1 to the second maximum value Max2 with a positive speed of variation (Speed+). The positive speed of variation (Speed+) may be predetermined. Alternatively, the positive speed of variation (Speed+) depends on the first response recorded. Preferably, the positive speed of variation (Speed+) is lower than or equal to, in absolute value, the first speed value, Speed1.

After reaching the second maximum value, Max2, the variable spherical power S decreases with a second speed value, Speed2, lower than the first speed value, Speed1. For instance the second speed value Speed2 is −5D/s.

Once again, the subject 5 presses the button as soon as he/she sees sharply the image of the visual stimulus 7. The second response of the subject 5 is entered at second time instant t₂ corresponding to a second sharp spherical power value, noted S2. The first sharp spherical power value, S1, may be different from the second sharp spherical power value, S2.

The continuous fluctuation of the spherical power S goes on the same way until finding a best focus (see FIG. 2). The best focus may be defined as the last sharp spherical power value, SN, determined by the subject, when the subject does not perceive any improvement in sharpness, and where N is an integer number preferably comprised between 2 and 10, and even more preferably between 2 and 5, or 2 and 4. Alternatively, the best focus may be derived from statistical or probabilistic processing based on the first, second, . . . and N^{th} sharp spherical power values S1, S2, . . . SN to determine the spherical power values surrounding the best focus value. The number N of fluctuation stages of the spherical power S may be predetermined. Alternatively, the number N depends on the response(s) recorded.

The responses S1, S2, . . . , SN are preferably recorded during a decrease in spherical power value S. Indeed, accommodation is not symmetric. When the optical power increases, the individual may compensate for a refraction error by accommodating. In contrast, when the optical power decreases from a Maximum value far enough from the best focus, the subject is less prone to accommodate.

During this eye exam process, the involvement of the subject 5 is minimal. The subject 5 only has to click when the image of the visual stimulus 7 (for example letters or optotypes) is seen the sharpest. The parameters of the spherical power change (Max, Min, Speed) are automatically induced by the control unit 3 and as a function of the subject's responses.

Preferably, during an eye exam process, the successive maximum values (Max1, . . . , Max(i), . . . MaxN) are decreasing as a function of time. Preferably also, the successive minimum values (Mint . . . , Min(i), . . . MinN) are increasing as a function of time. Preferably, the speed of variation (Speed i) between a maximum value (Max(i)) and the consecutive minimum value (Min(i)) depends on the in-between optical power value Si relative to a sharpness of the visual stimulus. The positive speed of variation between a minimum value Min(i) and the next maximum value Max(i+1) may be predetermined or be of the same absolute value or lower in absolute value and of opposite sign to the speed of variation (Speed i) between the maximum value Max(i) and the consecutive minimum value Min(i).

While varying the optical power value of the optical system 2, different kinds of visual stimulus 7 may be used. Conventional optotypes, Gabor patch or sinusoidal grating (with variable angle or not), or a lifestyle scene that include sufficiently high spatial frequencies to help detecting blur may be used. The range of high spatial frequencies may be between 10 and 60 cpd (cycles per degree), and preferably between 20 and 40 cpd.

Moreover, the visual stimulus 7 may be adapted during the eye exam as getting closer to the best focus SN. For example, the visual stimulus 7 displayed during the time interval between the first maximum value Max1 and the first minimum value Min1 comprises letters of large size corresponding to a visual acuity comprised between 1/10 and 5/10. The visual stimulus may be changed during the time interval between the first minimum value Min1 and the second maximum value Max2 so that the visual stimulus 7 displayed during the time interval between the second maximum value Max2 and the second minimum value Min2 comprises letters of smaller size. By gradually decreasing the size of the optotypes during the successive stages, the process enables to determine the best focus rapidly.

In another example, the visual stimulus 7 comprises a hybrid image 20 as illustrated on FIG. 3. In this case, decision-making is simplified. The subject 5 is not asked to respond when he/she sees the image 20 sharply or blurred, but when his/her perception changes from the first image 21 with low spatial frequencies (for example “I see the in-door scene”, see FIG. 4) to the second image 22 with high spatial frequencies (“Now I see the city”, see FIG. 5). More precisely, the first image 21 includes features having spatial frequencies below a low-pass cut-off frequency and, respectively, the second image 22 includes features having spatial frequencies at a high-pass cut-off frequency. Using different hybrid images 20 with various levels of high-pass cut-off frequency along the continuous refraction process, enables to get closer to the best visual perception without asking for decision about the sharpest perception of the image. For instance, the first stage is performed using a hybrid image with high spatial frequencies of 3 cycles/degree, and the hybrid image is change for the second stage so that it includes high spatial frequencies of 16 cycles/degree. The high spatial frequencies of the hybrid image may be increased until the individual perceives the high frequency image. The other advantage of hybrid images is that the subject 5 does not go further into negative powers with accommodation. Hybrid images are particularly suited for self-refraction processes because the subject's response is binary (the question being “Do I see another image?” for example, the second image with high spatial frequency) and does not require a comparison between two images to determine which one is perceived sharpest. During an eye examination process, the power variation of the optical system 2 remains continuous between Max1 and Min1, between Min1 and Max2, between Max2 and Min2 and so on. During the continuous power variation, the speed of variation varies and is controlled via the control unit 3.

Depending on the shape of the power variation, the speed of variation may vary either by steps or continuously.

In a second embodiment, the subject 5 adjusts himself the speed of variation and the direction of optical power change of the optical system 2.

The input device 8 is for example a dimmer or a joystick. The subject 5 uses for example the joystick to control the variable optical power. Pulling the joystick decreases the power. Pushing the joystick increases the power. The more the subject pushes or pulls the faster the power changes. The subject changes direction of power change as soon as he/she sees the visual stimulus with the best sharpness. In other words, the subject 5 adjusts the direction of power change and the speed of variation via the input device 8 (here a joystick) and the control unit 3 driving to the optical system 2. The best focus is obtained when the subject does not need to change the power anymore, that is when he/she reaches the best sharpness or, in other words, when this sharpness cannot be improved anymore.

In this case, we can prevent accommodation by adapting the visual stimulus 7 and the decision making to prevent the user from being trapped into a too negative power range. For example, a Gabor patch of large spatial frequencies is used during the first stage and then the subject is asked if the orientation of the first Gabor patch is detected (and not necessarily seen clear) and then, in case of a positive answer, increase the spatial frequencies of the Gabor patch along the process.

The first and second embodiments may be combined during a single eye exam process. For example, the first maximum power value Max1 may be predetermined. The subject controls the first speed of variation using a joystick, as detailed in the second embodiment. The subject enters the first response at first time instant t1 corresponding to the first spherical power value S1. Then, the control unit 3 determines the value of the second maximum power value Max2, and, possibly, the first minimum power value Mint, as detailed in the first embodiment. The control unit 3 sets the speed of variation from the first minimum power value Min1 up to the second maximum power value Max2. Then, the subject takes control again using the joystick to drive the second speed of variation between the second maximum power value Max2 and the second response at second time instant t2 with a second spherical power value S2.

In a variant of the second embodiment, the speed of variation is not controlled directly by the individual, but varies as a function of an input parameter, for example the reaction time of the individual. For example, the control unit 3 is interfaced with an apparatus for measuring an electro-encephalogram (or EEG) of the individual during the eye exam process, or with a pupil measurement system or with another reaction time measurement system. The apparatus for measuring delivers an EEG signal to the control unit 3. Alternatively, the pupil measurement system or the reaction time measurement system, delivers a pupil reaction signal, respectively a reaction time signal of the individual during the eye exam process. Reaction time may be evaluated by the subject's time to answer (keyed answer or oral answer) or by the pupil reaction time or by the EEG signal. The control unit 3 adjusts the speed of variation as a function of the reaction time of the individual.

In another embodiment, illustrated on FIG. 6, the temporal variation in spherical power S of the optical system 2 is sinusoidal, by parts, the sinusoid varying as a function of the subject's responses.

The variable spherical power S is initially set at a first maximum value, Max1, here of +20 diopters (D). This initial defocus of +20D prevents the subject from accommodating. More precisely, in a first stage, the spherical power S is sinusoidal between the first maximum value, Max1, and a first minimum value, Min1. The spherical power S may present a single oscillation or several oscillations between the first maximum value, Max1, and the first minimum value, Min1. The subject 5 enters for example a first response, at a first time instant t1 corresponding to a first spherical power value S1. The subject enters his/her response when he/she perceives the visual stimulus sharply (for example using optotypes) or when he/she perceives a change in the hybrid image perception, as explained above. Optionally, during further sinusoidal oscillations of the spherical power S, the subject enters a second response, during a second oscillation, corresponding to a second time instant t2 and a second spherical power value S2, and, respectively, a third response, during a third oscillation, corresponding to a third time instant t3 and a third spherical power value S3. The first amplitude of the sinusoidal oscillations during this first stage is equal to the difference between Max1 and Min1. The period of the sinusoidal oscillation corresponds to a first (temporal) frequency, for example comprised between 2 Hz and 4 Hz. The first amplitude and first frequency of the oscillations determine the first speed of variation between consecutive extremum values Max1 and Min1. In a second stage, the parameters of the sinusoidal variations of the spherical power S are changed: the amplitude of the oscillations is defined by a second maximum value, Max2, and a second minimum value, Min2, and the period of the sinusoidal oscillation increases corresponding to a lower second frequency, for example comprised between 1 Hz and 2 Hz. The parameters of the sinusoid in the second stage may be predetermined. Alternatively, in this third embodiment, the parameters of the sinusoidal power variations are changed according to the subject's response. In this example, the second amplitude and second frequency are function of spherical power value S1, S2 and/or S3 and/or of the first amplitude and first frequency. The second amplitude and second frequency of the oscillations determine the second speed of variation between consecutive extremum values Max2 and Min2. Thus, the second speed of variation of the optical power is adjusted according to the subject's response. Similarly, during sinusoidal oscillations of the spherical power S at the second frequency, the subject enters a fourth response corresponding to a fourth time instant t4 and a fourth spherical power value S4, and, possibly, a fifth response corresponding to a fifth time instant t5 and a fifth spherical power value S5. As illustrated on FIG. 6, in a third stage, the parameters of the sinusoidal variations of the spherical power S are changed again: for example the amplitude of the oscillations is defined by a third maximum value, Max3, and a third minimum value, Min3, and the period of the sinusoidal oscillation increases corresponding to a still lower third frequency, for example comprised between 0.5 Hz and 1 Hz. The third amplitude and third frequency are function of spherical power value S4 and/or S5 and/or of the second amplitude and second frequency. The third amplitude and third frequency of the oscillations determine the third speed of variation between consecutive extremum values Max3 and Min3. Thus, the third speed of variation of the optical power is adjusted according to the subject's response. During the third stage, the subject enters a sixth response corresponding to a sixth time instant t6 and a sixth spherical power value S6.

The third embodiment enables to determine the best focus or at least values surrounding the best focus. The third embodiment proposes several iterations, and thus several possibilities for the user to refine his appreciation of the best focus. Changes at each stage may be based on an average of several iterations at the previous stage(s), thus providing a more accurate result.

During the whole eye examination process, the optical power oscillations are slow enough to be perceived by the subject 5. In other words, the temporal frequency of a sinusoidal change in perception is within the range of detection and fusion limits of the eye 4 of the subject 5. Above 25 Hz, the subject 5 might fuse both maximum and minimum values of the optical power variations. Below 0.1 Hz, the subject 5 might miss to detect change of the stimulus. Advantageously, the temporal frequency is adjusted according to the spatial frequency of the stimuli (letters). For low spatial frequency stimuli (e.g. 0.5 cycle per degree (or cpd)), the temporal frequency of power change is preferably higher (e.g. 10 Hz) than for a high spatial frequency stimuli (e.g. 1 Hz).

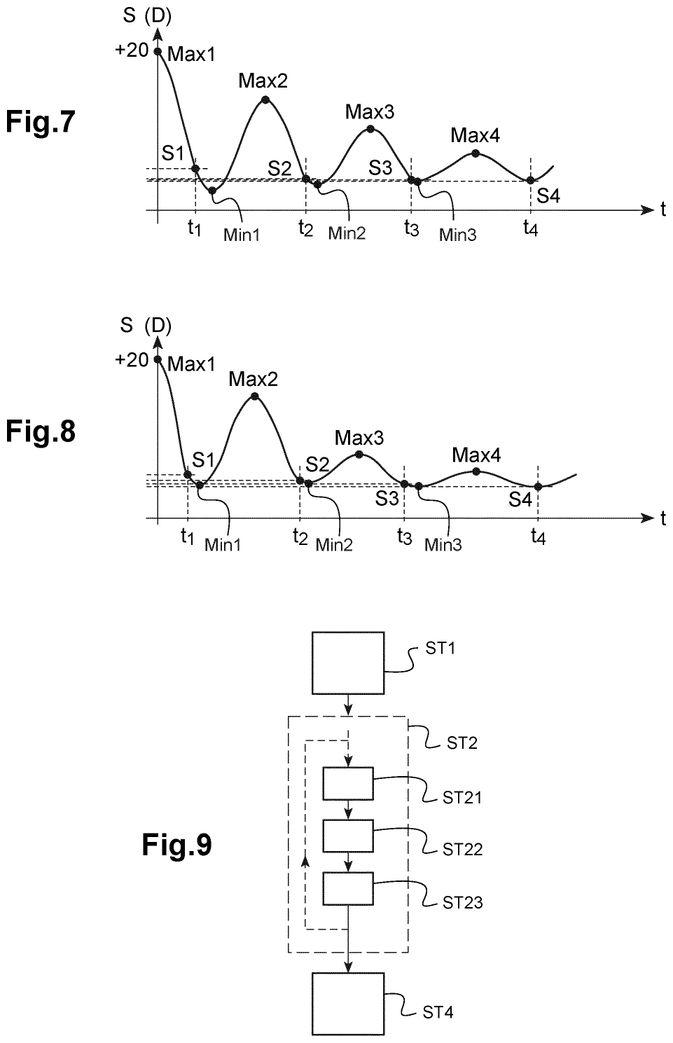

In another embodiment, illustrated on FIG. 7, the temporal variation in spherical power S of the optical system 2 is also sinusoidal by parts. In this embodiment, the parameters of the sinusoid are adjusted by the subject himself/herself. More precisely, the frequency of the sinusoid remains constant over the whole eye exam process, for instance 1 Hz, and the subject adjusts the amplitude of the sinusoidal curve. In the example illustrated on FIG. 7, this human-machine interaction can be achieved directly with a simple button or with a dimmer or with a joystick or even on the recording of the subject's brain signal informing about the level of blur/focus perceived by the subject. For example, on FIG. 7, in a first stage, the spherical power S is sinusoidal with a first amplitude between the first maximum value, Max1, and the first minimum value, Min1. The subject 5 enters a first response at a first time instant t1 corresponding to a first spherical power value S1 during a decreasing phase of the spherical power S. As illustrated on FIG. 7, the first stage comprises a single oscillation from Max1 to Mint Optionally, as described in relation with FIG. 6, the first phase at the first amplitude may comprise several oscillations. Then, the subject adjusts the sinusoidal curve to a second amplitude using for example the interface 8. The second maximum value, Max2, and the second minimum value, Min2, are calculated as a function of the second amplitude. Thus, the subject adjusts the second speed of variation of the optical power. The subject 5 enters a second response at a second time instant t2 corresponding to a second spherical power value S2 during a decreasing phase of the spherical power S. Similarly, the subject adjusts the sinusoidal curve at a third amplitude between a third, respectively fourth, maximum value, Max3, respectively Max4, and a third, respectively fourth, minimum value, Min3, respectively Min4, using for example the interface 8. Thus, the subject adjusts the third, respectively fourth, speed of variation of the optical power. The subject 5 enters a third, respectively fourth, response at a third, respectively fourth, time instant t3, respectively t4, corresponding to a third, respectively fourth, spherical power value S3, respectively S4, during a decreasing phase of the spherical power S. The fourth embodiment enables to determine the best focus, S4, or at least values S1, S2, S3, S4 surrounding the best focus.

The fourth embodiment is quicker than the other embodiments described above. Moreover, it provides a good compromise between easiness and adaptation of the process to the subject since only amplitude is to be controlled by the subject.

In a fifth embodiment, illustrated on FIG. 8, the temporal variation in spherical power S of the optical system 2 is also sinusoidal by parts. In this embodiment, the parameters of the sinusoid are adjusted by the subject himself/herself. More precisely, the subject adjusts both amplitude and frequency of the sinusoidal curve during the eye exam process. As in the previous embodiments, this human-machine interaction can be achieved directly with a simple button or with a dimmer or with a joystick or even on the recording of the subject's brain signal informing about the level of blurriness/sharpness perceived by the subject. For example, on FIG. 8, in a first stage, the spherical power S is sinusoidal with a first frequency and a first amplitude between the first maximum value, Max1, and the first minimum value, Min1. The subject 5 enters a first response at a first time instant t1 corresponding to a first spherical power value S1 during a decreasing phase of the spherical power S. As illustrated on FIG. 8, the first stage comprises a single oscillation from Max1 to Mint Optionally, as described in relation with FIG. 6, the first phase at the first amplitude and first frequency may comprise several oscillations. Then, the subject adjusts the sinusoidal curve to a second frequency and a second amplitude using for example the interface 8. Thus, the subject adjusts the second speed of variation of the optical power. Preferably, the second frequency is lower than the first frequency. The second maximum value, Max2, and the second minimum value, Min2, are calculated as a function of the second amplitude. The subject 5 enters a second response at a second time instant t2 corresponding to a second spherical power value S2 during a decreasing phase of the spherical power S. Similarly, the subject adjusts the sinusoidal curve to a third frequency and a third amplitude, respectively fourth frequency and a fourth amplitude, between a third, respectively fourth, maximum value, Max3, respectively Max4, and a third, respectively fourth, minimum value, Min3, respectively Min4, using for example the interface 8. The subject 5 enters a third, respectively fourth, response at a third, respectively fourth, time instant t3, respectively t4, corresponding to a third, respectively fourth, spherical power value S3, respectively S4, during a decreasing phase of the spherical power S. Thus, the subject adjusts the third, respectively fourth, speed of variation of the optical power. Preferably, the third frequency is lower than the second frequency and the fourth frequency is lower than the third frequency. The fifth embodiment enables to determine the best focus, S4, or at least values S1, S2, S3, S4 surrounding the best focus.

The fifth embodiment provides a fully personalized process, wherein the subject adapts both amplitude and frequency to tiny appreciate the best focus position. Thus, he/she has a perfect control of what he/she can see. The fifth embodiment may be quicker than the first, second and third embodiments.

In the embodiments illustrated on FIGS. 6 to 8, the variations in optical power are sinusoidal or sinusoidal by parts. Other periodic continuous variations of the optical power may be contemplated without departing from the scope of the invention. Alternatively, the optical power varies in a pseudo-periodic way: the amplitude and frequency of variation in optical power are adjusted and selected so as to optimize perception of change according to the subject. For instance, an initial power variation is applied with an initial frequency and initial amplitude. Then the subject is asked to adjust firstly the frequency (reduced frequency or increased frequency), for which he perceives the more easily the variations in power. In a second step, the subject is asked to reduce the amplitude to adapt the minimum and maximum values to get closer to the desired or best perceived corrective value or to choose the amplitude to optimize the change in perception. Thus, the speed of variation is selected in amplitude and frequency so as to optimize perception of change in optical power feature by the eye of the subject. In this last configuration variations in optical power may follow any other kind of time function that may be adapted to the subject. The subject determines which point is the correct one. The initial amplitude may be adapted to the sensitivity of the subject or the subject may self-adapt the amplitude and frequency of variations in optical power.

In a variant of any of the embodiments disclosed herein, the first maximum power value Max1 and/or the first minimum power value Min1 may be determined according to an objective starting value (for example resulting from an auto-refraction measurement or depending on the current correction of the subject) and/or according to the age of the subject.

In an example according to any one of the embodiments disclosed herein, we may finish the process with a duochrome visual test. More precisely, this step corresponds to the assessment given by the subject during, for example, a red/green duochrome test. During this test, the subject is presented with an image comprising black optotypes displayed on a red background on one side and black optotypes displayed on a green background on the other side. The subject then indicates his answer, for example “better vision on red background” or “better vision on green background”. If the subject has a better vision of the optotypes on the red background, the sphere value S should be decreased, and if he has a better vision of the optotypes shown on the green background, the sphere value S should be increased. Alternatively or complementarily, we may finish the process with a visual comfort appreciation on letters chart.

FIG. 9 represents schematically a block diagram of a method for measuring refraction of a subject according to the present disclosure.

At step ST1, the optical power features of an optical system are set to initial determined values. For example, the optical power of the optical system is set at the first maximum value, Max1.

Step ST2 comprises several steps ST21, ST22, ST23, which may be repeated iteratively.

At step ST21, the optical power feature is varied continuously at a determined (first, respectively second, third . . . ) speed of variation starting from the determined value (first, respectively second, third . . . maximum).

Step ST22 includes recording a response of the subject relatively to a sharpness of the visual stimulus seen through the optical system while continuously varying the optical power feature.

Step ST23 includes adjusting the speed of variation as a function of the response of the subject recorded at previous step ST22. Preferably, Step ST23 also includes determining values of a consecutive minimum value, (Min1, Min2), consecutive maximum value (Max2, Max3, . . . ), and consecutive speed of variation (Speed2, Speed3 . . . ) as a function of the response of the subject recorded at previous step ST22.

The process includes repeating the steps ST21 to ST23 by a predetermined number N of iterations, where N is an integer comprised preferably between two and five, or until a best focus is determined.

Step ST4 includes determining at least one visual refraction feature of the subject based on the response(s) of the subject at the previous step(s) ST21 to ST23.

The above examples have been described in relation with spherical power variations to determine spherical correction needed by the eye 4 of the subject 5.

A similar process may be applied to determine cylinder and/or axis for a correction of astigmatism.

In this case, the visual stimulus 7 may comprise non-oriented features or comprises for example two oriented Gabor patches at two angles teta1 and teta2, for example with teta2=teta1+45 degrees. The optical system 2 comprises an optical system which allows continuous astigmatism variation, for example motorized cross-cylinders as used in Vision-R800 instrument. The astigmatism is for example decomposed in well-known cylindrical power vectorial components along any orientations of teta1 and teta2. For example, we use the couple (J0, J45) and the two values of the vectorial components (J0, J45) are tested at these two orientations.

Or, we can test cylinder power, noted C, and cylinder axis, A, alternatively. The process for determining astigmatism may start from an initial objective measurement or with an initial estimation of sphere or with an arbitrary (0.5D) cylinder value if no cylinder. Any of the embodiments described above for varying sphere S can apply to each of the astigmatism component C or A (or respectively J0 or J45).

Although representative processes and articles have been described in detail herein, those skilled in the art will recognize that various substitutions and modifications may be made without departing from the scope of what is described and defined in the appended claims.

The process(es) described above may be implemented in a phoropter having an optical system with a continuously variable power feature. For instance, the process(es) may be implemented in Vision-R800 instrument.

Advantageously, the processes wherein the subject self-adjusts the speed of variation may be used for self-refraction for example in a in self-service refraction system.

Advantageously, the processes wherein an operator or another instrument requiring an operator controls the speed of variation are driven by an eye-care professional (ECP) practicing refraction.

1. An apparatus for determining at least one visual refraction feature of a subject by showing a visual stimulus to the subject, the apparatus comprising:
an optical system arranged on an optical path between an eye of the subject and the visual stimulus,
the optical system being adapted to provide an optical power that is continuously variable as a function of time; and
a control unit for driving the optical power of the optical system and an input device adapted for recording a response of the subject relative to a sharpness of the visual stimulus seen through the optical system,
the control unit being adapted to adjust a speed of variation of the optical power as a function of the response recorded.

2. The apparatus according to claim 1, wherein the control unit is adapted to adjust the speed of variation by going through predetermined stages as a function of the response recorded.

3. The apparatus according to claim 2, wherein the control unit is adapted to drive the optical power of the optical system at an initial maximum positive value (Max1), to decrease the optical power from the initial maximum positive value (Max1) to a first optical power value (S1) relative to a first sharpness (Sharp1) of the visual stimulus, with a first speed of variation (Speed1), wherein the input device is adapted for recording a first response of the subject relative to the first sharpness (Sharp1), and wherein the control unit (3) is adapted to increase the optical power to a second maximum value (Max2) below the initial maximum positive value (Max1).

4. The apparatus according to claim 3, wherein the control unit is adapted to decrease the optical power from the first optical power value (S1) relative to the first sharpness (Sharp1) of the visual stimulus until a first minimum value (Min1) depending on the first optical power value (S1) relative to the first sharpness of the visual stimulus (Sharp1), before increasing the optical power to the second maximum value (Max2).

5. The apparatus according to claim 3, wherein the second maximum value (Max2) depends on the first optical power value (S1) relative to the first sharpness (Sharp1) of the visual stimulus.

6. The apparatus according to claim 2, wherein the control unit is adapted to implement a continuous variation of the optical power between N successively decreasing maximum values (Max1, . . . , Max(i), MaxN) and N successively increasing minimum values (Min1, . . . , Min(i), . . . MinN), where N is an integer comprised between 2 and 5, wherein the speed of variation (Speed i) between one of said maximum values (Max(i)) and a consecutive minimum value (Min(i)) depends on the in-between optical power value relative to a sharpness (Sharp i) of the visual stimulus, and wherein the following maximum value (Max(i+1)) also depends on the said in-between optical power value relative to a sharpness (Sharp i) of the visual stimulus.

7. The apparatus according to claim 1, wherein the optical power includes a spherical power, a cylindrical power and cylinder axis and/or an addition power and/or a binocular balance between both eyes of the subject.

8. The apparatus according to claim 1, further comprising a calculator adapted to determine said at least one visual refraction feature of the subject as a function of one or a plurality of responses of the subject.

9. The apparatus according to claim 1, wherein the control unit is adapted to select the visual stimulus depending on the current predetermined stage of variation of the speed and/or depending on the response recorded.

10. The apparatus according to claim 1, wherein the input device comprises a user interface adapted to record an input parameter and wherein the control unit is adapted to drive the speed of variation as a function of the input parameter.

11. The apparatus according to claim 10, wherein the user interface comprises a button, a dimmer, a joystick, a device adapted to record a physiological signal of the subject, a voice recognition system, a computer interface, a brain-computer interface with electrodes recording brain activity in real-time, an interface with a pupil measurement system or with a reaction time measurement system, a tracking movement or eyetracking system and/or a face or hand or body expression analyzing system.

12. The apparatus according to claim 1, the apparatus being adapted to record a reaction time of the subject.

13. The apparatus according to claim 1, wherein a range of the optical power and/or a range of the speed of variation are preselected as a function of data relative to the subject and/or as a function of distance to the visual stimulus and/or the optical power variation is periodic, pseudo periodic or non periodic.

14. A system for measuring at least one visual refraction feature of a subject, the system comprising:
the apparatus according to claim 1; and
an objective refraction measurement device and/or a device for measuring micro-fluctuations of refraction of the eye, adapted for providing preliminary measurements, the control unit being adapted to define an initial profile for the speed of variation of the optical power according to said preliminary measurements.

15. A method for determining at least one visual refraction feature of a subject, the method comprising:
a) varying continuously an optical power of an optical system in a phoropter, the optical system being arranged on an optical path between an eye of the subject and a visual stimulus;
b) recording a response of the subject to the continuous variation in optical power of the optical system, the response being relative to a sharpness of the visual stimulus seen through the optical system with continuously variable optical power;
c) adjusting a speed of variation of the optical power as a function of the response recorded; and
d) repeating the steps a) to c) until a best focus is determined.

16. The apparatus according to claim 4, wherein the second maximum value (Max2) depends on the first optical power value (S1) relative to the first sharpness (Sharp1) of the visual stimulus.

17. The apparatus according to claim 3, wherein the control unit is adapted to implement a continuous variation of the optical power between N successively decreasing maximum values (Max1, . . . , Max(i), . . . MaxN) and N successively increasing minimum values (Min1, . . . , Min(i), MinN), where n is an integer comprised between 2 and 5, wherein the speed of variation (Speed i) between one of said maximum values (Max(i)) and a consecutive minimum value (Min(i)) depends on the in-between optical power value relative to a sharpness (Sharp i) of the visual stimulus, and wherein the following maximum value (Max(i+1)) also depends on the said in-between optical power value relative to a sharpness (Sharp i) of the visual stimulus.

18. The apparatus according to claim 4, wherein the control unit is adapted to implement a continuous variation of the optical power between N successively decreasing maximum values (Max1, . . . , Max(i), . . . MaxN) and N successively increasing minimum values (Min1, . . . , Min(i), . . . MinN), where N is an integer comprised between 2 and 5, wherein the speed of variation (Speed i) between one of said maximum values (Max(i)) and a consecutive minimum value (Min(i)) depends on the in-between optical power value relative to a sharpness (Sharp i) of the visual stimulus, and wherein the following maximum value (Max(i+1)) also depends on the said in-between optical power value relative to a sharpness (Sharp i) of the visual stimulus.

19. The apparatus according to claim 3, wherein the optical power includes a spherical power, a cylindrical power and cylinder axis and/or an addition power and/or a binocular balance between both eyes of the subject.

20. The apparatus according to claim 4, wherein the optical power includes a spherical power, a cylindrical power and cylinder axis and/or an addition power and/or a binocular balance between both eyes of the subject.
